(19) United States
(12) Patent Application Publication
(10) Pub. No.: US 2010/0135856 A1
(43) Pub. Date: Jun. 3, 2010

(54) NANOPARTICLE FOR DETECTING BIOMATERIALS AND BIOSENSOR BY USING THE NANOPARTICLE

(75) Inventors: Hyeon-Bong PYO, Daejeon (KR); Moon-Youn Jung, Daejeon (KR); Seon-Hee Park, Daejeon (KR)

Correspondence Address: AMPACC Law Group, 3500 188th Street S.W., Suite 103, Lynnwood, WA, 98037, US

(73) Assignee: Electronics and Telecommunications Research Institute, Daejeon (KR)

(21) Appl. No.: 12/482,223

(22) Filed: Jun. 10, 2009

(30) Foreign Application Priority Data
Nov. 29, 2008 (KR) 10-2008-0120188

(51) Int. Cl. G01N 33/543; B32B 5/00; G01N 33/544; G01N 33/551; G01N 33/552; G01N 33/553

(52) U.S. Cl. 422/69; 428/403; 436/518; 436/529; 436/524; 436/527; 436/525

(57) ABSTRACT

Provided are a nanoparticle for detecting biomaterials and a biosensor by using the nanoparticle. The nanoparticle includes a metal nanostructure around which an electric field is induced by localized surface plasmon resonance when light is irradiated onto a surface of the metal nanostructure, a spacer covering the surface of the metal nanostructure, and capture molecules specifically reacting with fluorophore-labeled target molecules, and immobilized on a surface of the spacer.

### CROSS-REFERENCE TO RELATED APPLICATIONS

This U.S. non-provisional patent application claims priority under 35 U.S.C. §119 of Korean Patent Application No. 10-2008-120188, filed on Nov. 29, 2008, the entire contents of which are hereby incorporated by reference.

### BACKGROUND OF THE INVENTION

The present invention disclosed herein relates to a nanoparticle for detecting biomaterials and a biosensor by using the nanoparticle, and more particularly, to a nanoparticle for detecting biomaterials and a biosensor by using the nanoparticle, which can improve the detection efficiency by enhancing the intensity of an optical signal during detection of the biomaterials.

Biosensors are a device capable of detecting an optical or electrical signal that varies with the selective reaction and combination between a biological receptor recognizing a specific biomaterial and an analyte to be analyzed. That is, biosensors can detect the existence of specific biomaterials, or analyze biomaterials qualitatively or quantitatively. Here, the biological receptor (i.e., capture molecule) includes nucleic acid, protein, cell, tissue, enzyme, antibody, DNA, etc., which can selectively react and bind to a specific material. To detect a signal, biomaterials are detected and analyzed by using various physicochemical methods, for example, a method of detecting a change in electrical signal according to whether an analyte exists or not, or a method of detecting a change in an optical signal caused by chemical reaction between a receptor and an analyte.

Meanwhile, an optical biosensor using a change in optical signal employs a label detecting method where a specific antibody or antigen is labeled with a radioactive isotope or fluorophore, and then the specific antigen is quantitatively measured using a variation of radiation or fluorescent intensity occurring during the reaction between the labeled antigen and the specific antibody.

A biosensor (for example, a fluorescence microscope) detecting an optical signal from a biomaterial detects and analyzes the biomaterial using fluorescence emitted from a fluorophore when an incident light having a wavelength equal to an absorption wavelength of the fluorophore labeled on an antibody or antigen is irradiated onto a sample including the biomaterial. The fluorophore absorbs light of a specific wavelength out of light provided from an external light source, and then emits light of a specific wavelength according to physical and chemical properties.

### SUMMARY OF THE INVENTION

The present invention provides a nanoparticle that can enhance the intensity of an optical signal to detect biomaterials.

The present invention also provides a biosensor with further improved detection efficiency using a nanoparticle that can enhance the intensity of an optical signal to detect biomaterials.

The subject of the present invention is not limited to the aforesaid, but other subjects not described herein will be clearly understood by a person with ordinary in the art from descriptions below.

Embodiments of the present invention provide nanoparticles for detecting biomaterials, including: a metal nanostructure around which an electric field is induced by localized surface plasmon resonance when light is irradiated onto a surface of the metal nanostructure; a spacer covering the surface of the metal nanostructure; and capture molecules specifically reacting with fluorophore-labeled target molecules, and immobilized on a surface of the spacer.

In other embodiments of the present invention, biosensors include: a biomaterial reacting unit in which nanoparticles are provided, each nanoparticle including a metal nanostructure around which an electric field is induced by localized surface plasmon resonance when light is irradiated onto a surface of the metal nanostructure, a spacer covering the surface of the metal nanostructure, and capture molecules specifically reacting with fluorophore-labeled target molecules, and immobilized on a surface of the spacer; a light-emitting unit providing incident light to the nanoparticles; and a light-receiving unit detecting an emission light that is emitted from the fluorophore of the nanoparticle by the incident light and enhanced by the localized surface plasmon resonance

Particularities of other embodiments are included in the detailed description and drawings.

### BRIEF DESCRIPTION OF THE FIGURES

The accompanying figures are included to provide a further understanding of the present invention, and are incorporated in and constitute a part of this specification. The drawings illustrate exemplary embodiments of the present invention and, together with the description, serve to explain principles of the present invention. In the figures:

FIG. 1 is a graph illustrating absorption and emission spectra used in detecting biomaterials;

FIG. 2 illustrates a nanoparticle for detecting biomaterials according to an embodiment of the present invention;

FIG. 3 is a graph illustrating an extinction efficiency versus a resonance wavelength when gold nanoparticles exist in a dielectric material;

FIG. 4 illustrates a nanoparticle for detecting biomaterials according to another embodiment of the present invention;

FIG. 5 is a graph illustrating an extinction efficiency versus a resonance wavelength when core-shell nanoparticles exist in a dielectric material;

FIG. 6 is a graph illustrating extinction efficiencies of a near-field and a far-field of a core-shell nanoparticle;

FIG. 7 illustrates a biosensor using a nanoparticle for detecting biomaterials according to an embodiment of the present invention;

FIG. 8 illustrates a binding structure of sandwich immunoassay in which nanoparticles bind to target molecules in the biosensor of FIG. 7; and

FIG. 9 illustrates a biosensor using a nanoparticle for detecting biomaterials according to another embodiment of the present invention.

### DETAILED DESCRIPTION OF PREFERRED EMBODIMENTS

Advantages and features of the present invention, and implementation methods thereof will be clarified through following embodiments described with reference to the accompanying drawings. The present invention may, however, be embodied in different forms and should not be construed as limited to the embodiments set forth herein. Rather, these embodiments are provided so that this disclosure will be thorough and complete, and will fully convey the scope of the present invention to those skilled in the art. Further, the present invention is only defined by scopes of claims. Like reference numerals refer to like elements throughout.

In the present disclosure, the technical terms are used only for explaining a specific exemplary embodiment while not limiting the present invention. The terms of a singular form may include plural forms unless referred to the contrary. The meaning of “include,” “comprise,” “including,” or “comprising,” specifies a property, a region, a fixed number, a step, a process, an element and/or a component but does not exclude other properties, regions, fixed numbers, steps, processes, elements and/or components.

Additionally, the embodiment in the detailed description will be described with sectional and/or plan views as ideal exemplary views of the present invention. In the drawings, the dimensions of layers and regions are exaggerated for clarity of illustration. Therefore, areas exemplified in the drawings have general properties, and are used to illustrate a specific shape of a device. Thus, this should not be construed as limited to the scope of the present invention.

In the present disclosure, target molecules, which are biomolecules exhibiting specific attributes, may be interpreted as analytes, and correspond to antigens in embodiments of the present invention.

In the present disclosure, capture molecules, which are biomolecules specifically binding to target molecules, may be interpreted as probe molecules, receptors, or acceptors, and correspond to capture antibodies in embodiments of the present invention.

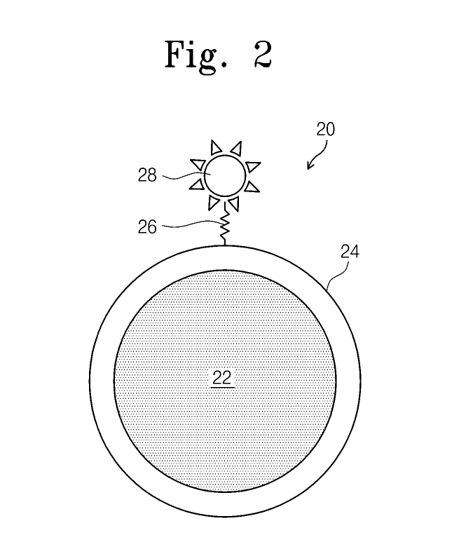

Hereinafter, a nanoparticle for detecting biomaterials according to an embodiment of the present invention will be described in detail with reference to FIG. 2.

FIG. 2 illustrates a nanoparticle 20 for detecting biomaterials according to an embodiment of the present invention.

Referring to FIG. 2, the nanoparticle 20 for detecting biomaterials according to this embodiment of the present invention may include a metal nanoparticle 22, a spacer 24 covering a surface of the metal nanoparticle 22, and capture molecules immobilized on a surface of the spacer 24.

A localized surface plasmon resonance phenomenon occurs on a surface of the metal nanoparticle 22 and inside the metal nanoparticle 22 due to light of a specific wavelength that is supplied from the outside. A resonance wavelength may vary with a material (e.g., composition, thickness or refractive index of a dielectric material) surrounding the surface of the metal nanoparticle 22. Therefore, biomaterials immobilized on the surface of the metal nanoparticle 22 can be detected and quantified using a variation of surface plasmon resonance wavelength.

The spacer 24 surrounds the surface of the metal nanoparticle 22, and separates a fluorophore 28 from the metal nanoparticle 22 by a predetermined distance. Linkers 26 may be immobilized on the surface of the spacer 24, and the fluorophores 28 may directly or indirectly bind to the linkers. The linkers 26 immobilized on the surface of the spacer 24 can facilitate the immobilization of the fluorophore or an analyte material labeled with the fluorophore.

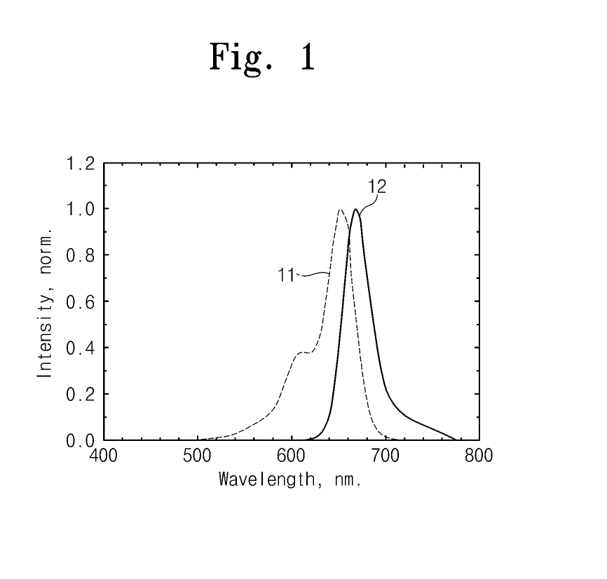

In the fluorophore 28 directly or indirectly bound to the spacer 24, a wavelength difference (called Stokes' shift) between an absorption light 11 and an emission light 12 is very small, for example, in a range of 20 nm to 30 nm, as illustrated in FIG. 1. Furthermore, the intensity of the emission light 12 is very lower than that of the absorption light 11. Thus, a fluorescent signal emitted from the fluorophore 28 should be enhanced to increase the detection sensitivity of biomaterials.

The spacer 24 may separate the fluorophore 28 from the surface of the metal nanoparticle 22 by a predetermined distance in order that the energy of an emission light emitted from the fluorophore 28 may not be transferred to the metal nanoparticle 22 when the energy of scattered light generated from the metal nanoparticle 22 is transferred to the fluorophore 28. To be specific, the spacer 24 can transfer the energy of the scattered light, which is emitted while a localized surface plasmon resonance phenomenon occurs on the surface of the metal nanoparticle 22, to the fluorophore 28 while not quenching the energy of the emission light emitted from the fluorophore 28. Therefore, when light of a specific wavelength (resonance wavelength of the metal nanoparticle) is irradiated onto the nanoparticle 20, the energy of a fluorescent signal generated from the fluorophore 28 can be maximized.

Even in the case where capture molecules (e.g., antibodies) are immobilized on the surface of the spacer 24, a thickness of the spacer 24 can be adjusted so as to optimize the energy transfer from the metal nanoparticle 22 to the fluorophore 28, making it possible to maintain a predetermined distance between the fluorophore 28 and the metal nanoparticle 22. Herein, the capture molecules immobilized on the surface of the spacer 24 include materials that can selectively and specifically bind to target molecules which are to be detected and analyzed. For example, the capture molecules may include protein, cell, virus, nucleic acid, organic molecule or inorganic molecule. The protein may include any biomaterial such as antigen, antibody, matrix protein, enzyme, and coenzyme. The nucleic acid may include DNA, RNA, PNA, LNA, or hybrids thereof.

In more detail, since the movement of free electrons existing on the surface of and inside the metal nanoparticle 22 is limited by the size of the metal nanoparticle 22, a surface plasmon phenomenon occurs when an electromagnetic wave (i.e., energy or wavelength) is applied from the outside.

The surface plasmon phenomenon refers to the quantized oscillation of electron density caused by the polarization of the electrons existing inside the metal nanoparticle 22 when light of a specific wavelength is irradiated on the surface of the metal nanoparticle 22. A change of a dielectric material adsorbed on the surface of the metal nanoparticle 22 induces a wavelength shift of a surface plasmon wave.

In addition, a localized surface plasmon resonance phenomenon refers to the excitation of surface plasmon of the metal nanoparticle 22 by light of a specific wavelength that is absorbed and scattered by the metal nanoparticle 22 when the light impinges on (or illuminates) the surface of the metal nanoparticle 22. The localized surface plasmon resonance phenomenon differently occurs according to a kind, composition, size, shape of nanoparticles and an ambient material surrounding the nanoparticles. Furthermore, light of a specific wavelength is absorbed in the surface of the metal nanoparticle 22, and light of a specific wavelength is scattered according to an ambient material surrounding the surface of the metal nanoparticle 22. Therefore, if a variation of surface plasmon resonance wavelength is detected by sensing a scattered light emitted from the surface of the metal nanoparticle 22, the changes in thickness or refractive index of a material adsorbed on the surface of the metal nanoparticle 22 can be detected and analyzed.

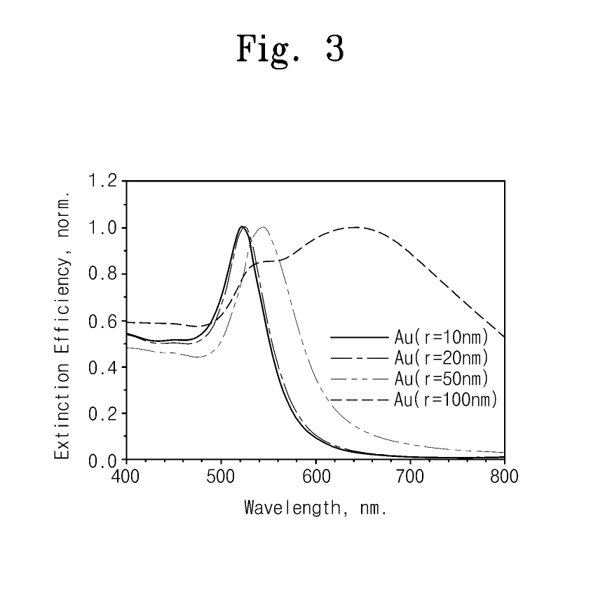

In FIG. 3, a graph shows extinction efficiency versus a wavelength according to a size of a metal nanoparticle. Specifically, FIG. 3 is a graph illustrating extinction efficiency according to a variation of a resonance wavelength with different size of when gold nanoparticles in a dielectric material.

Referring to FIG. 3, it can be seen that the resonance wavelength of the metal nanoparticle varies with the size of the metal nanoparticle. That is, it can be seen that the resonance wavelength becomes longer as the size of the metal nanoparticle increases.

The metal nanoparticle 22, in which the localized surface plasmon resonance phenomenon occurs, may include a noble metal of which a dielectric function has a minus imaginary part, for example, gold (Au) or silver (Ag).

An electric field is formed around the metal nanoparticle 22 by virtue of the localized surface plasmon resonance. In a near-field around the metal nanoparticle 22, the intensity of a scattered light exponentially increases as it gets closer to the surface of the metal nanoparticle 22. At this time, when the fluorophore 28 existing in the near-field receives such an enhanced energy that it can be excited from the metal nanoparticle 22, the intensity of the emission light (i.e., fluorescent signal) emitted from the fluorophore 28 can be considerably increased.

Further, as the fluorophore 28 gets closer to the surface of the metal nanoparticle 22 within a predetermined distance (about 10 nm) or less in the near-field around the metal nanoparticle 22, a non-radiative energy transfer occurs from the fluorophore 28 to the metal nanoparticle 22 so that the emission light of the fluorophore 28 is quenched. That is, when a distance between the fluorophore 28 and the metal nanoparticle 22 is reduced to the predetermined distance or less, the energy of the scattered light transferred from the metal nanoparticle 22 to the fluorophore 28 can be transferred to the metal nanoparticle 22 again. Thus, the energy of the light emitted from the fluorophore 28 may be quenched. Specifically, an energy transfer rate (k_{T}) from the fluorophore 28 to the metal nanoparticle 22 is expressed as Equation (1).

$${k}_{T} = ( \frac{1}{{τ}_{D}} ) \times {( \frac{{r}_{0}}{r} )}^{6} \qquad ( 1 )$$

where k_{T} is energy transfer rate, r is a distance between the fluorophore 28 and the metal nanoparticle 22, τ_{D} is a fluorescence decay time of the fluorophore 28, and r₀ is a Förster distance.

From Equation (1), if the distance (r) between the fluorophore 28 and the metal nanoparticle 22 is smaller than the Förster distance (r₀), the energy transfer rate (k_{T}) is sharply increased, and thus the intensity of the emission light is quenched after all.

Therefore, the spacer 24 with a predetermined thickness may be formed on the surface of the metal nanoparticle 22 so as to increase the intensity of the emission light from the fluorophore according to the localized plasmon resonance, and not to quench the intensity of the emission light by maintaining the distance between the fluorophore 28 and the metal nanoparticle 22 constantly.

Meanwhile, when the resonance wavelength of the metal nanoparticle is equal to an absorption wavelength of the fluorophore, the energy of the light emitted from the fluorophore can be maximized. Thus, a core-shell nanoparticle having a broad tunable range of the resonance wavelength is used in another embodiment of the present invention.

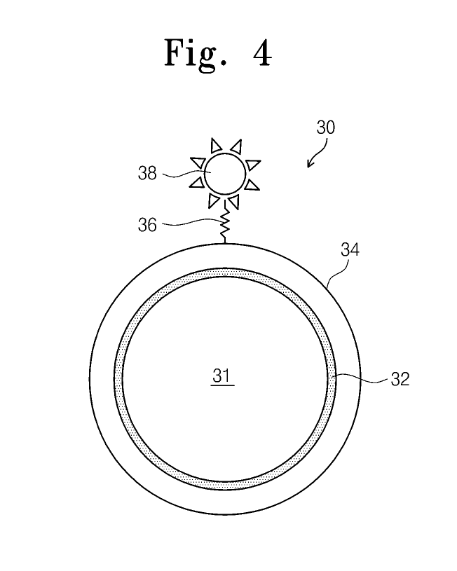

FIG. 4 illustrates a nanoparticle for detecting biomaterials according to another embodiment of the present invention. Referring to FIG. 4, in the nanoparticle 30, a nano-sized dielectric particle 31 is coated with a metal nano-thin film 32, and a surface of the metal nano-thin film 32 is covered with a spacer 34 adjusting a distance between a fluorophore 38 and the metal nano-thin film 32.

The dielectric particle 31 may be a solid dielectric material such as SiO₂, TiO₂ and Ta₂O₅, or the dielectric particle 31 may be liquid or gas, e.g., air or water. For example, the dielectric particle 31 may be a silica core.

The metal nano-thin film 32 may be a metal thin film having a thickness of several nanometers to several hundreds of nanometers. For example, the metal nano-thin film 32 may include gold (Au) or silver (Ag).

The spacer 34 surrounds the surface of the metal nano-thin film 32, and capture molecules or linkers 36 are immobilized on the surface of the spacer 34. The capture molecules or linkers 36 may directly or indirectly bind to the fluorophore 38. That is, the spacer 34 separates the fluorophore 38 from the surface of the metal nano-thin film 32 by a predetermined distance in order to reduce the non-radiatve energy transfer occurring from the fluorophore 38 to the metal nano-thin film 32 in a near-field formed around the metal nano-thin film 32 due to the resonance phenomenon.

The spacer 34 should have a structure that can be easily immobilized on the surface of the metal nano-thin film 32, has uniform spatial distribution and surface orientation, and can easily functionalize the surface thereof. For example, the spacer 34 may include a self-assembled monolayer (SAM), human serum albumin (HSA), polyethylene glycol, or dextran. A chain of an organic molecule or an inorganic molecule may also be used as the spacer 34.

The spacer 34 made of the chain of the organic molecule or the inorganic molecule may include the linkers 36 attached to the surface thereof. The spacer 34 prevents a distance between the fluorophore 38 and the metal nano-thin film 32 from being reduced to a predetermined value or less. Therefore, it is possible to reduce the energy transferred from the fluorophore 38 to the metal nano-thin film 32.

In more detail, according to another embodiment of the present invention, the metal nano-thin film 32 is formed on the surface of the dielectric nanoparticle 31 to increase a tunning range of the plasmon resonance wavelength in the metal nanoparticle 30. That is, the nanoparticle 30 has a core-shell structure. In the case where the nanoparticle has the core-shell structure, the dielectric exists inside the metal nano-thin film 32 and on an external surface of the metal nano-thin film 32, and therefore the movement of free electrons may be further limited inside the metal nano-thin film 32 rather than in the metal nanoparticle (see 22 in FIG. 2).

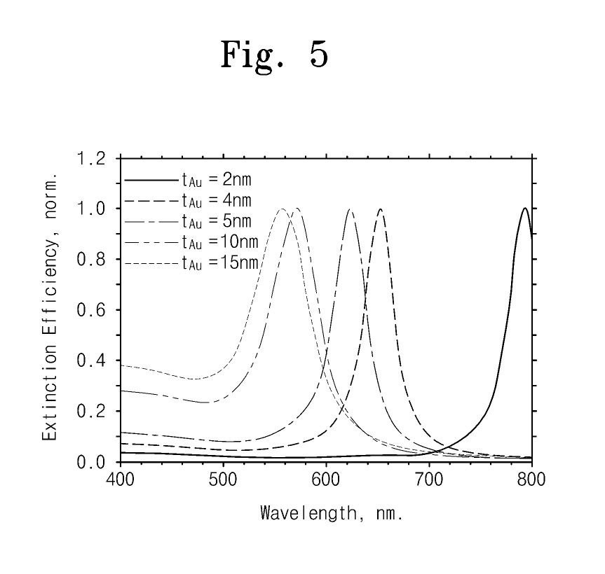

In FIG. 5, a graph shows extinction efficiency versus wavelength according to a thickness of a metal nano-thin film 32. Specifically, the graph of FIG. 5 is plotted according to a function of an extinction efficiency of core-shell nanoparticles dispersed in water with respect to the wavelength. Herein, the extinction efficiency is defined as the summation of absorption efficiency and scattering efficiency.

Referring to FIG. 5, it can be seen that the extinction efficiency varies with the thickness of a gold nano-thin film surrounding the surface of the silica nanoparticle with a diameter of about 30 nm. That is, from the comparison of the graphs in FIGS. 3 and 5, it can be understood that a variation range of the plasmon resonance wavelength is greater in the core-shell nanoparticle (see 32 in FIG. 4) rather than the metal nanoparticle (see 22 in FIG. 2) provided that the nanoparticles are equal in size. Therefore, a tunning range of the plasmon resonance wavelength is great in the core-shell nanoparticle (see 32 in FIG. 4).

Meanwhile, when the wavelength of light incident on the nanoparticle 30 is equal to the plasmon resonance wavelength, the energy of a scattered light generated from the metal nano-thin film 32 is transferred to the fluorophore 38, thereby maximizing the energy of emission light.

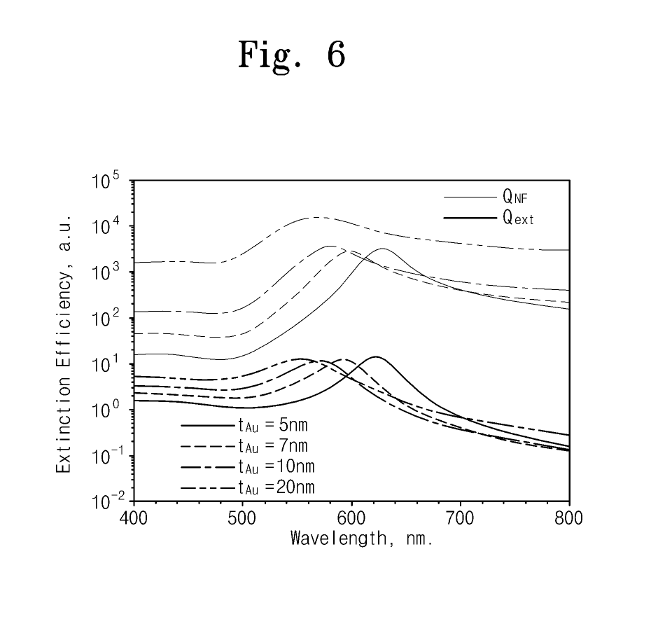

In FIG. 6, a graph shows comparison results of a near-field (QNF) and far-field extinction efficiency (Qext) resulting from the localized surface plasmon resonance phenomenon. FIG. 6 is a graph illustrating an extinction efficiency of each nanoparticle composed of a silica core having a diameter of 30 nm and a gold shell (i.e., gold nano-thin film) having a thickness of 5, 7, 10 or 20 nm.

Referring to FIG. 6, it can be seen that the intensity of energy detected from the surface of the gold nano-thin film is stronger in a near-field than a far-field due to the localized surface plasmon resonance phenomenon.

At the same time, when the fluorophore 38 existing in the near-field approaches the surface of the metal nanoparticle 32 within a predetermined distance (about 10 nm) or less, the non-radiative energy transfer occurs from the fluorophore 38 to the metal nanoparticle 32 so that the emission light of the fluorophore 38 is quenched.

Accordingly, the spacer 34 is formed on the surface of the metal nano-thin film 32 such that the energy transferred from the surface of the metal nano-thin film 32 can not only enhance the intensity of the emission light emitted from the fluorophore 38 but also maintain a distance between the fluorophore 38 and the metal nano-thin film 32 constantly not to quench the intensity of the emission light when the localized surface plasmon resonance phenomenon occurs on the surface of the metal nano-thin film 32. That is, the spacer 34 constantly maintains the distance between the fluorophore 34 and the surface of the metal nano-thin film 32 to a predetermined distance.

The thickness of the spacer 34 is determined in consideration of two contrary effects, i.e., the effect of increasing the energy of electric field due to the surface plasmon resonance of the metal nano-thin film 32, and the quenching effect caused by the non-radiative energy transfer from the fluorophore 38 to the metal nano-thin film 32. Thus, the thickness of the spacer 34 may be defined as a thickness of a single thin film existing on the surface of the metal nano-thin film 32. Alternatively, the thickness of the spacer 34 may be a thickness corresponding to the total length of the capture molecule, the target molecule, and the linker 36 disposed between the fluorophore 38 and the surface of the metal nano-thin film 32. In other words, the capture molecule, the target molecule, and the linker 36 disposed between the fluorophore 38 and the surface of the metal nano-thin film 32, are positioned in the electric field where the non-radiative energy transfer may occur from the fluorophore 38 to the metal nano-thin film 32 again.

Herebelow, a biosensor capable of detecting and analyzing target molecules using the nanoparticles that can enhance the intensity of the emission light emitted from the fluorophore will be described.

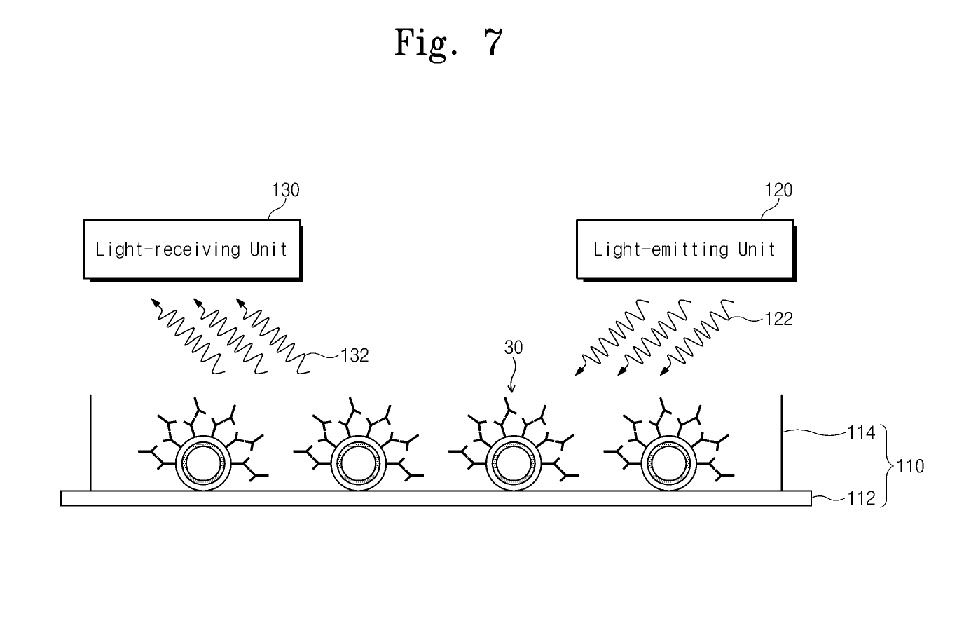

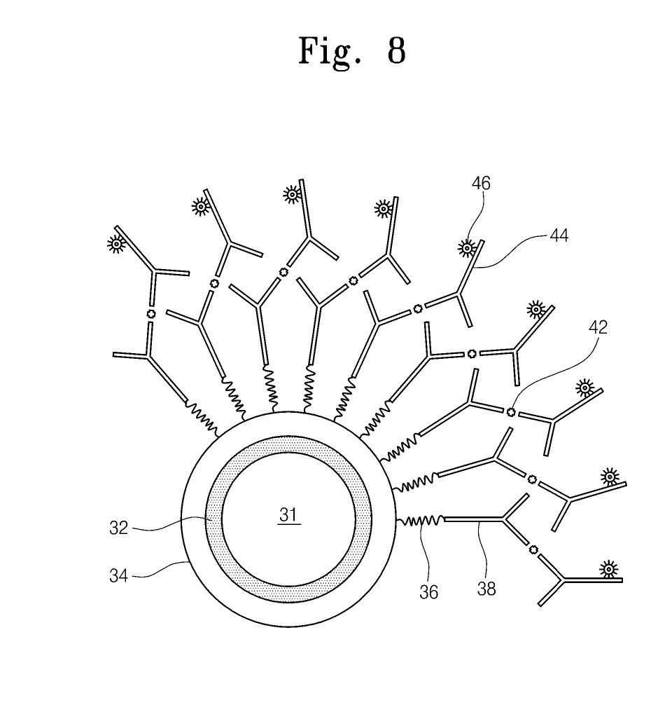

FIG. 7 illustrates a biosensor according to an embodiment of the present invention. FIG. 8 illustrates a binding structure of sandwich type of immunoassay in which nanoparticles bind to target molecules in the biosensor of FIG. 7.

Referring to FIG. 7, a biosensor may include a biomaterial reacting unit 110, a light-emitting unit 120, and a light-receiving unit 130.

The biomaterial reacting unit 110 may include a substrate 112, a reaction chamber 114 formed on the substrate 112.

The substrate 112 may be formed of a transparent material allowing light to be transmitted. For instance, the substrate 112 may include one of a plastic substrate, a glass substrate and a silicon substrate. Also, the substrate 112 may be formed of a polymer such as PDMS (polydimethylsiloxane), PMMA (polymethylmethacrylate), PC (polycarbonate), COC (cyclic olefin copolymer), PA (polyamide), PE (polyethylene), PP (polypropylene), PPE (polyphenylene ether), PS (polystyrene), POM (polyoxymethylene), PEEK (polyetheretherketone), PTFE (polytetrafluoroethylene), PVC (polyvinylchloride), PVDF (polyvinylidene fluoride), PBT (polybutyleneterephthalate), FEP (fluorinated ethylenepropylene), and PFA (perfluoralkoxyalkane).

The reaction chamber 114 receives nanoparticles 30 specifically binding to the target molecules to be analyzed. The nanoparticles 30 may be uniformly distributed in an aqueous ambient of a liquid phase, for example, water, buffer solution, plasma, serum, or the like. The localized surface plasmon resonance phenomenon occurs at the surface of the surface of the metal nano-thin film 32 or the metal nanoparticle when light of a specific wavelength is irradiated onto the biosensor.

Specifically, the nanoparticle 30 may be a core-shell nano particle of which a surface is surrounded by a spacer, as illustrated in FIG. 8. The core-shell nanoparticle includes a dielectric nanoparticle 31 whose a surface is surrounded by a metal nano-thin film 32. The core-shell nanoparticle has a broad range of resonance wavelength of the localized surface plasmon resonance phenomenon that occurs at the surface of the metal nano-thin film 32 when light of a specific wavelength is incident from the outside. Accordingly, it is possible to tune the resonance wavelength to the absorption wavelength of the fluorophore. The nanoparticle 30 may be a metal nanoparticle having a surface surrounded by the spacer.

The spacer 34 maintains a distance between a fluorophore 46 and the metal nanoparticle or metal nano-thin film 32 such that the energy of the scattered light may be transferred to the fluorophore 46 when the surface plasmon resonance occurs. Immunoreactions between antigens and antibodies may occur at the surface of the spacer 34, and thus target molecules 42 can be detected by analyzing a fluorescence signal emitted from the fluorophore 46 according to the immunoreactions.

More specifically, FIG. 8 illustrates a binding structure of a capture molecule and a target molecule via sandwich type of immunoreactions at the surface of the nanoparticle 30. As illustrated in FIG. 8, the binding structure resulting from sandwich type of immunoreactions will be described in the embodiments of the present invention. That is, a capture antibody 38 immobilized on the surface of the spacer 34 is used as a capture molecule, and an antigen 42, i.e., target molecule, binds to a detection antibody 44 labeled with the fluorophore 46. The detection antibody 44 specifically binds to the antigen, and a site of the detection antibody 44 bound to the antigen differs from a site where the capture antibody 38 and the antigen bind.

In detail, the capture molecules 38 specifically binds to the target molecules 42 to be detected and analyzed are immobilized on the surface of the spacer 34. The capture molecules 38 may include protein, cell, virus, nucleic acid, organic molecule or inorganic molecule. The protein may include any biomaterial such as antigen, antibody, matrix protein, enzyme, and coenzyme. The nucleic acid may include DNA, RNA, PNA, LNA, or hybrids thereof. In this embodiment of the present invention, the capture molecule 38 may be a capture antibody specifically binding to the target molecule (antigen) 42 to be analyzed. The capture antibody 38 specifically binds to the antigen to be analyzed, and the antigen may be immobilized on the surface of the spacer 34.

The capture antibody 39 immobilized on the surface of the spacer 34 can be more strongly immobilized on the surface of the spacer 34 through the linker 36. A method of immobilizing the capture body 38 onto the surface of the spacer 34 may include chemical adsorption, covalent-binding, electrostatic attraction, copolymerization, avidin-biotin affinity system, and the like.

To be specific, to immobilize the capture antibody 38 to the surface of the spacer 34, a functional group may be introduced to the surface of the spacer 34. For example, a functional group such as carboxyl group (—COOH), thiol group (—SH), hydroxyl group (—OH), silane group, amine group, and epoxy group may be introduced to the surface of the spacer 34.

The target molecule 42, which is a material obtained from a living body, may provided to the biomaterial reacting unit 10 in a state that it is contained in a solution. That is, body fluids obtained from the living body, for example, blood, serum, plasma, urine, or salvia are provided to the biomaterial reacting unit 110, where the target molecules 42 are contained in the body fluids. The target molecules 42 may include nucleic acid, cell, virus, protein, organic molecule or inorganic molecule. The protein may include any biomaterial such as antigen, antibody, matrix protein, enzyme, and coenzyme. The nucleic acid may include DNA, RNA, PNA, LNA, or hybrids thereof.

The target molecules 42 may bind to the capture molecules 38 in the biomaterial reacting unit 10 through chemical and biochemical reactions such as nucleic acid hybridization, antigen-antibody reaction, and enzyme-linked reaction.

Before the target molecules 42 are provided to the biomaterial reacting unit 110, the target molecules 42 may be labeled with the fluorophore 46. That is, the target molecules 42 directly or indirectly bind to the fluorophores 46 and are then provided into the biomaterial reacting unit 110. In this embodiment of the present invention, the fluorophore 46 may be a fluorophore that emits light in a wavelength range from about 400 nm to 800 nm.

A body fluid may include not only the target molecules 42 to be detected but also other molecules which also bind to the target molecules 42 non-specifically. Therefore, the target molecules 42 specifically binds to the capture molecules 38 are labeled with the fluorophore 46, and then provided to the biomaterial reacting unit 110.

The labeling of the target molecules 42, i.e., antigens with the fluorophore 46 may be directly or indirectly performed. That is, the fluorophore 46 may directly bind to the antigen 42. Furthermore, a site-specific binding to the antigen 42 allows the fluorophore 46 to bind to a detection antibody 44 which differs from the capture antibody 38, and also allows the detection antibody 44 to which the fluorophore 46 is labeled, bind to the antigen 42. Binding of linker 36-capture antibody 38-antigen 42-detection antibody 44-fluorophore 46 may occur at the surface of the spacer 34 due to the specific binding between the antibody and antigen. In such a binding structure, a distance between the metal nano-thin film 32 and the fluorophore 46 may correspond to the summation of the thickness of the spacer 34 and the lengths of the linker 36, capture antibody 38, antigen 42 and detection antibody 44. At this time, the spacer 34, the linker 36, the capture antibody, the antigen 42 and the detection antibody 44 prevent the distance between the fluorophore 46 and the metal nano-thin film 32 from being reduced to a predetermined distance or less. Consequently, it is possible to reduce the energy transfer of emission light from the fluorophore 46 to the metal nano-thin film 32.

Referring to FIG. 7 again, the light-emitting unit 120 irradiates an incident light of a specific wavelength onto the biomaterial reacting unit 110, that is, nanoparticles of the biomaterial reacting unit 110. The light-emitting unit 120 may be adjusted such that the incident light 122 has an incident angle of about 45° with respect to a substrate 112 of the biomaterial reacting unit 110.

The light-emitting unit 120 may include a xenon lamp which emits a polychromatic light. In the case of using the xenon lamp as a light source, the light-emitting unit 120 further includes an optical filter to provide monochromatic light of which a wavelength is equal to the plasmon resonance wavelength of the nanoparticles 30. Alternatively, the light-emitting unit 120 may include a laser diode which emits a monochromatic light having a wavelength equal to the plasmon resonance wavelength.

The light-receiving unit 130 is installed nomal to the substrate 112, and detects the emission light (fluorescence) emitted from the fluorophore 46 to thereby detect and analyze the target molecules 42 binding to the nanoparticles 30. For example, the light-receiving unit 130 may include a photodiode, an ocular lens, a charge coupled device (CCD) or a CMOS image sensor.

As such, when light is irradiated from the light-emitting unit 120 emits toward the nanoparticles 30 after the capture molecules 38 and the target molecules 42 react with each other at the surfaces of the nanoparticles in the biomaterial reacting unit 110, the light-receiving unit 130 can detect an enhanced fluorescent signal from the fluorophore 46.

Hereinafter, a biosensor according to another embodiment of the present invention will be described.

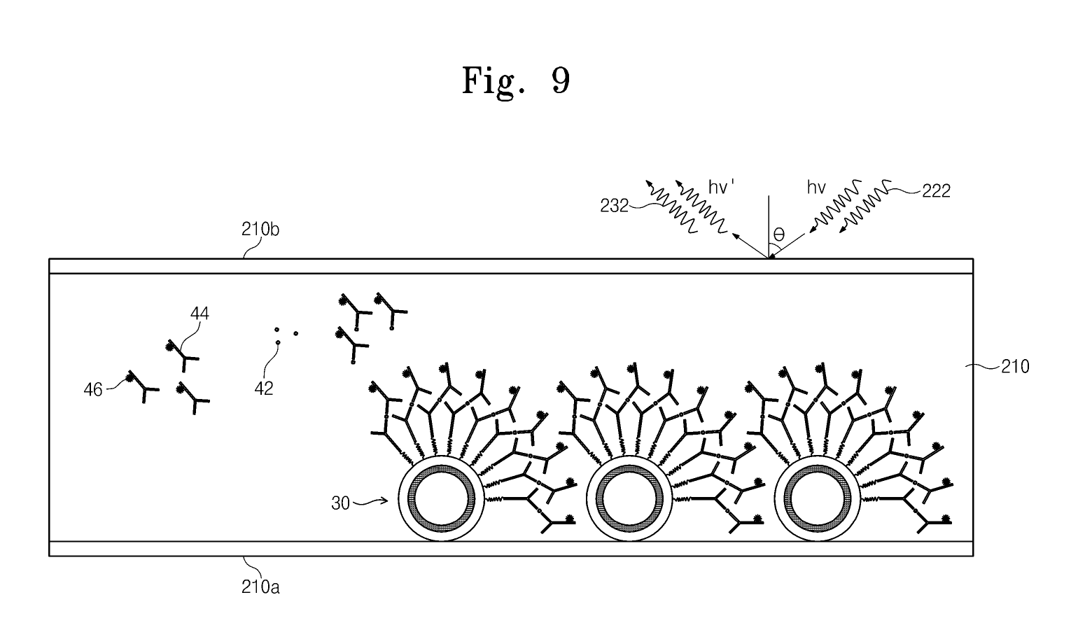

Referring to FIG. 9, a biosensor according to this embodiment includes a microfluidic channel 210 in which capture molecules 38 and target molecules 42 react with each other. An incident light 222 is provided to the microfluidic channel 210, and the target molecules can be detected and analyzed by detecting an emission light 232 emitted from the surface of the nanoparticle 30 by the incident light 222.

In more detail, the biosensor according to this embodiment of FIG. 9 includes the microfluidic channel formed by a lower substrate 210a and an upper substrate 210b, which are spaced apart by a predetermined distance. The microfluidic channel 210 corresponds to the biomaterial reacting unit (see 110 in FIG. 7) illustrated in the previous embodiment of FIG. 7.

The lower and upper substrates 210a and 210b, which form the microfluidic channel 210, may include glass, silica, silicon, PMMA, polycarbonate (PC), polystyrene (PS), or cyclic olefin copolymer (COC). The lower and upper substrates 210a and 210b may be transparent or opaque, and exhibit black or white. Kinds or colors of the lower and upper substrates 210a and 210b may differ from or equal to each other.

The nanoparticles 30 on which the capture molecules 38 are immobilized and specifically bind to the target molecules 42, are immobilized on the substrate 210a, or dispersed in the microfluidic channel 210.

Further, a solution containing the target molecules 42 that bound specifically to fluorophore-labeled detection antibody 44 may be supplied to the microfluidic channel 210. And the target molecules 42 may bind to the capture molecules 38 immobilized on the nanoparticles 30 by the immunoreactions.

According to the present invention, a nanoparticle for detecting a biomaterial includes a spacer on a surface of a metal nanostructure such that a distance between a fluorophore and the metal nanostructure is optimized. Accordingly, the fluorophore can receive energy from the metal nanostructure while not quenching the intensity of light emitted from the fluorophore, thus enhancing the intensity of a fluorescent signal emitted from the fluorophore. Consequently, biomaterials can be detected and analyzed by detecting an enhanced fluorescent signal emitted from the fluorophore, which makes it possible to improve the detection efficiency of a biosensor.

The above-disclosed subject matter is to be considered illustrative, and not restrictive, and the appended claims are intended to cover all such modifications, enhancements, and other embodiments, which fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. A nanoparticle for detecting biomaterials, comprising:
a metal nanostructure around which an electric field is induced by localized surface plasmon resonance when light is irradiated onto a surface of the metal nanostructure;
a spacer covering the surface of the metal nanostructure; and
capture molecules specifically reacting with fluorophore-labeled target molecules, and immobilized on a surface of the spacer.

2. The nanoparticle of claim 1, wherein the spacer prevents non-radiative energy transfer from the fluorophore to the metal nanostructure when light is irradiated onto the fluorophore.

3. The nanoparticle of claim 2, wherein the spacer is formed of self-assembled monolayer (SAM), human serum albumin (HSA), polyethylene glycol (PEG), or dextran.

4. The nanoparticle of claim 1, wherein the metal nanostructure comprises a metal nanoparticle or a core-shell nanoparticle formed of a metal nano-thin film covering a surface of a dielectric core.

5. The nanoparticle of claim 4, wherein the dielectric core is formed of a dielectric material in a solid, liquid or gaseous state.

6. The nanoparticle of claim 4, wherein the dielectric core is formed of SiO₂, TiO₂, Ta₂O₅, air or water.

7. The nanoparticle of claim 4, wherein the metal nanoparticle and the metal nano-thin film is formed of gold (Au) or silver (Ag).

8. A biosensor comprising:
a biomaterial reacting unit in which nanoparticles are provided, each nanoparticle comprising a metal nanostructure around which an electric field is induced by localized surface plasmon resonance when light is irradiated onto a surface of the metal nanostructure, a spacer covering the surface of the metal nanostructure, and capture molecules labeled with a fluorophore specifically reacting with fluorophore-labeled target molecules, and immobilized on a surface of the spacer;
a light-emitting unit providing incident light to the nanoparticles; and
a light-receiving unit detecting an emission light that is emitted from the fluorophore of the nanoparticle by the incident light and enhanced by the localized surface plasmon resonance.

9. The biosensor of claim 8, wherein the spacer prevents non-radiative energy transfer from the fluorophore to the metal nanostructure when light is irradiated onto the fluorophore.

10. The biosensor of claim 9, wherein the spacer is formed of self-assembled monolayer (SAM), human serum albumin (HSA), polyethylene glycol (PEG), or dextran.

11. The biosensor of claim 8, wherein the metal nanostructure comprises a metal nanoparticle or a core-shell nanoparticle formed of a metal nano-thin film covering a surface of a dielectric core.

12. The biosensor of claim 11, wherein the metal nanoparticle and the metal nano-thin film is formed of gold (Au) or silver (Ag).

13. The biosensor of claim 8, wherein the biomaterial reacting unit comprises a substrate and a reaction chamber that is formed on the substrate and receives the nanoparticles, or comprises a microfluidic channel formed by an upper substrate and a lower substrate that are spaced apart from each other by a predetermined distance.

14. The biosensor of claim 13, wherein the substrate comprises a plastic substrate, a glass substrate or a silicon substrate.

15. The biosensor of claim 8, wherein the incident light has a wavelength equal to a localized surface plasmon resonance wavelength of the nanoparticle.

16. The biosensor of claim 15, wherein the light-emitting unit comprises:
a light source emitting polychromatic light; and
an optical filter transmitting only light, which has a wavelength equal to a wavelength of an emission light emitted from the fluorophore, to provide the transmitted light as the incident light.

17. The biosensor of claim 8, wherein the capture molecules are immobilized by a carboxyl group (—COOH), a thiol group (—SH), a hydroxyl group (—OH), a silane group, an amine group (—NH2) or an epoxy group.

18. The biosensor of claim 8, wherein the capture molecules or the target molecules comprise at least one selected from the group consisting of an nucleic acid, a cell, a virus, a protein, an organic molecule and an inorganic molecule.

19. The biosensor of claim 18, wherein the nucleic acid comprises at least one selected from the group consisting of DNA, RNA, PNA, LNA, and hybrids thereof.

20. The biosensor of claim 18, wherein the protein comprises at least one selected from the group consisting of an enzyme, a substrate, an antigen, an antibody, a ligand, an aptamer, and a receptor.
